(19) United States
(12) Patent Application Publication
(10) Pub. No.: US 2006/0222592 A1
(43) Pub. Date: Oct. 5, 2006

(54) Nanoparticles and methods of manufacturing nanoparticles for electronic and non-electronic applications

(76) Inventor: Clemens Burda, Cleveland Heights, OH (US)

Correspondence Address: BRINKS HOFER GILSON & LIONE, P.O. BOX 10395, CHICAGO, IL, 60610, US

(21) Appl. No.: 11/099,206

(22) Filed: Apr. 5, 2005

(51) Int. Cl. A61K 49/00; H01L 51/00

(52) U.S. Cl. 424/9.6; 257/40; 438/1; 977/890; 977/927

(57) ABSTRACT

Binary or ternary nanoparticles containing a group I metal, a group VI non-metal, and perhaps a group III, IV, or V non-metal are produced using a single or multiple source precursor. A precursor and a surfactant are mixed, a solvent is added to the mixture, the mixture is heated at a temperature close to the boiling point of the solvent for a desired amount of time, and the mixture is cooled. The nanoparticles are separated from the solvent by permitting the solvent to evaporate. The resulting nanoparticles may be used in photovoltaic and other semiconductor applications, imaging, biological applications, or in compositions.

### FEDERALLY SPONSORED RESEARCH OR DEVELOPMENT

The present application was made in part with Government support (Grant No. CHE-0239688 awarded by the National Science Foundation). The Government may have certain rights in this application.

### FIELD

The present application relates to nanoparticles. More specifically, the present application relates to binary and ternary nanoparticles, methods of manufacturing the nanoparticles, and various applications in which the nanoparticles are used.

### BACKGROUND

Nanotechnology will be a key technology in the 21^{st} century. This technology is focused on the ability to produce materials in the nanometer range. These materials have great potential in many different applications such as electronic, optoelectronic or other materials applications. In the future, these materials may underpin many new materials compositions or electronics related devices that will be incorporated into a vast array of consumer goods.

Within the field of nanotechnology, the production of nanoparticles (or quantum dots) has been the subject of increased investigations in recent years because of their many potential applications. In particular, because of their size, quantum dots can take advantage of the laws of quantum physics which can be advantageous for many applications. More specifically, a wide range of optical and electrical properties may be tailored to a particular application by altering the composition, nature and size of the nanoparticle. For example, nanoparticles offer great promise in the development of photovoltaic devices with improved efficiencies.

Conventional synthesis of nanoparticles with different structures has been somewhat problematic. Synthesis has been achieved by various chemical and electrochemical methods. However, the synthesis is usually a challenging task and not easily amenable to scaling up to the volumes used in manufacturing processes, due to cost and space considerations for example. Moreover, the resulting nanoparticles may be of relatively poor quality with many defects or have a wide, uncontrollable variation in quality. Presently, there is an inability to control the size, shape, and other characteristics of these nanoparticles in a cost efficient manner.

Therefore, there is a need for an improved nanoparticle compositions and methods of producing same.

### SUMMARY

Accordingly, new compositions for nanoparticles with improved properties and new methods of forming nanoparticles are disclosed and claimed.

According to one aspect of the invention a novel composition for nanoparticles is provided. This composition for the nanoparticles comprises an element selected from a group I metal; an element selected from a group VI non-metal; and an element selected from a group III non-metal, a group IV non-metal or a group V non-metal.

According to another aspect of the invention another novel composition for nanoparticles is provided. This composition for the nanoparticles comprises an element selected from a group I metal; and an element selected from selenium or selenium.

In another aspect of the invention a solution containing a plurality of nanoparticles is provided. The nanoparticles in the solution comprise an element selected from a group I metal and an element from a group VI non-metal. The average separation between adjacent nanoparticles in the solution is at least about 10 nm or at least about 100 nm.

A novel method for manufacturing nanoparticles is also provided. According to this method, a plurality of precursors is combined with a surfactant. A solvent is added to the combination of the precursors and surfactant to form a mixture. The mixture is then heated. The heated mixure is then cooled thereby forming nanoparticles.

In another aspect of the invention, another method for manufacturing nanoparticles is provided. According to this method a single precursor, containing selenium or tellurium, is combined with a surfactant. A solvent is added to the combination to form a mixture. The mixture is heated to decompose the single precursor. The heated mixture is then cooled to form nanoparticles.

In other aspects of the invention, methods of using the nanoparticles include forming photovoltaic devices that include the nanoparticles and/or imaging molecular materials such as biomolecular imaging, biomedical imaging, and imaging non-organic molecular materials.

### BRIEF DESCRIPTION OF THE DRAWINGS

FIGS. 1 and 2 are transmission electron microscope (TEM) images of Cu_{1.8}S.

FIG. 3 is a TEM image of the size and shape evolution of CuInSe₂ nanoparticles at 1 min, 5 min, 20 min, 60 min, and 14 hrs from left to right.

FIGS. 4-8 are TEM images of CuInSe₂ nanoparticles at 1 min, 5 min, 20 min, 60 min, and 14 hrs, respectively.

FIG. 9 is a ultraviolet (UV)-visible absorption and photoluminescence (PL) spectra of CuInSe₂ quantum dots.

FIG. 10 is a UV-visible absorption of CuInSe₂ at different reaction times: 1 min, 5 min, 20 min, 60 min, and 14 hr.

FIG. 11 is an X-ray diffraction pattern of CuInSe₂ and the JCPDS pattern of 80-0535.

FIG. 12 is a TEM image of the CuInS₂ particles.

FIG. 13 is a UV-visible PL spectra of Cu_{1.8}S and Cu₂O nanoparticles.

FIG. 14 is the transient absorption spectra of Cu_{1.8}S quantum dots in which the inset shows trapping under excitation power.

FIG. 15 is a nanosecond decay curve and picosecond rise time of the laser-induced transient absorption monitored at 600 nm.

FIG. 16 shows the relaxation dynamics of the transient absorption observed at 500 nm in a time window up to 80 ps.

FIG. 17 is the transient absorption spectra of Cu₂O quantum dots.

FIG. 18 shows the decay dynamics of Cu₂O quantum dots.

FIGS. 19a and 19b show the relaxation time for copper sulfide nanoparticles and a diagram of hot electron trapping, respectively.

FIGS. 20a and 20b show trap state lifetimes for copper sulfide nanoparticles and a diagram of electron-hole recombination at the traps, respectively.

FIG. 21 shows spheres encapsulating nanoparticles.

FIG. 22 illustrates a method of encapsulating nanoparticles in the spheres.

FIG. 23 shows ultrasound-mediated destruction of spheres.

FIGS. 24(a) and (b) are optical micrographs of undoped bubbles and undoped spheres, respectively.

FIGS. 25(a) and (b) are fluorescence image of bubbles and spheres doped with CdTe nanoparticles, respectively.

FIG. 26 illustrates the use of nanoparticles in PDT/drug delivery systems.

### DETAILED DESCRIPTION OF THE EMBODIMENTS

The invention contemplates nanoparticles with improved characteristics and methods for making such nanoparticles. The nanoparticles of the present invention may have a composition, as described in more detail below, which is a binary structure that includes selenium or tellurium. This binary structure may be made by single or multiple precursor methods as described below.

Also contemplated by the invention are novel nanoparticles that have a novel ternary structure. This ternary structure may also be made by single or multiple precursor methods as described below.

Throughout this description and in the appended claims, the definitions that are provided in the section below labeled “Definitions” to be understood.

The nanoparticles described herein may be formed from different binary, ternary, or quaternary (or larger) systems.

In more detail, according to one novel aspect of the invention, binary nanoparticles may comprise an element selected from a group I metal and an element selected from a group VI non-metal, and in particular selenium or tellurium. For example, experimental results are discussed below for binary nanoparticles such as copper sulfide CuₓS (x=1, 1.8, 2 . . . ) and copper oxide. Other binary nanoparticles such as CuSe, CuTe, AlS, AlSe, AlTe, or similar nanoparticles using Ag or Au for example, may also be manufactured. Aluminum may be preferable as it is non-toxic and relatively cheap.

In more detail, according to another novel aspect of the invention, ternary nanoparticles may contain an element selected from a group I metal, an element selected from a group VI non-metal, and an element selected from a group III, IV, or V non-metal. Such ternary nanoparticles include, for example CuInSe₂, and CuInS₂. Similar to the above, CuInTe₂, CuAlS₂, or CuAsS₂ may be manufactured using the single or multiple source precursor method. Other group I metals such as Al, Ag, or Au can replace Cu in ternary nanoparticles.

The average diameter of the binary or ternary nanoparticles ranges from about 0.1 nm to about 1000 nm. In some embodiments, the average diameter ranges from about 0.3 nm to about 500 nm. In some embodiments, the average diameter ranges from about 0.5 nm to about 350 nm. In some embodiments, the average diameter ranges from about 1 nm to about 200 nm.

Good quality nanoparticles with controllable characteristics and properties may be manufactured using a single or multiple source precursor method. The various nanoparticles are formed by combining particular elements and forming nanocrystals from the elements. In one example, the nanoparticles are formed by combining one or more precursors with a surfactant. A solvent is then added to the combination of the precursors and surfactant to form a mixture. The mixture is then heated, preferably near the boiling point of the solvent, for a predetermined amount of time and the heated mixture is cooled to form nanoparticles. After the mixture is cooled, the nanoparticles are separated from the solvent, for example, by evaporating the solvent or distillation of the nanoparticles. The precursors may be, for example, commercial grade metal precursors. If a single precursor is used, the precursor may be synthesized before combining it with the surfactant.

In one example, nanoparticles of the digenite phase of copper sulfide (Cu_{1.8}S) were synthesized by using a single source precursor method. A single source precursor was synthesized using solvents, such as analytical grade solvents, to create a dithiocarbamate precursor Cu(S₂CNEt₂)₂. The dithiocarbamate precursor was prepared by reacting Na(S₂CNEt₂) with a copper salt. NaS₂CNEt₂×3H₂O (31.2 mmol) was added into an ethanol solution of CuCl₂ (14.8 mmol) under constant stirring at room temperature for 15 minutes. The resulting solution was concentrated by vacuum evaporation and the dithiocarbamate complex extracted with toluene. Subsequent filtration and evaporation of the excess solvent afforded a copper complex with a 95% yield.

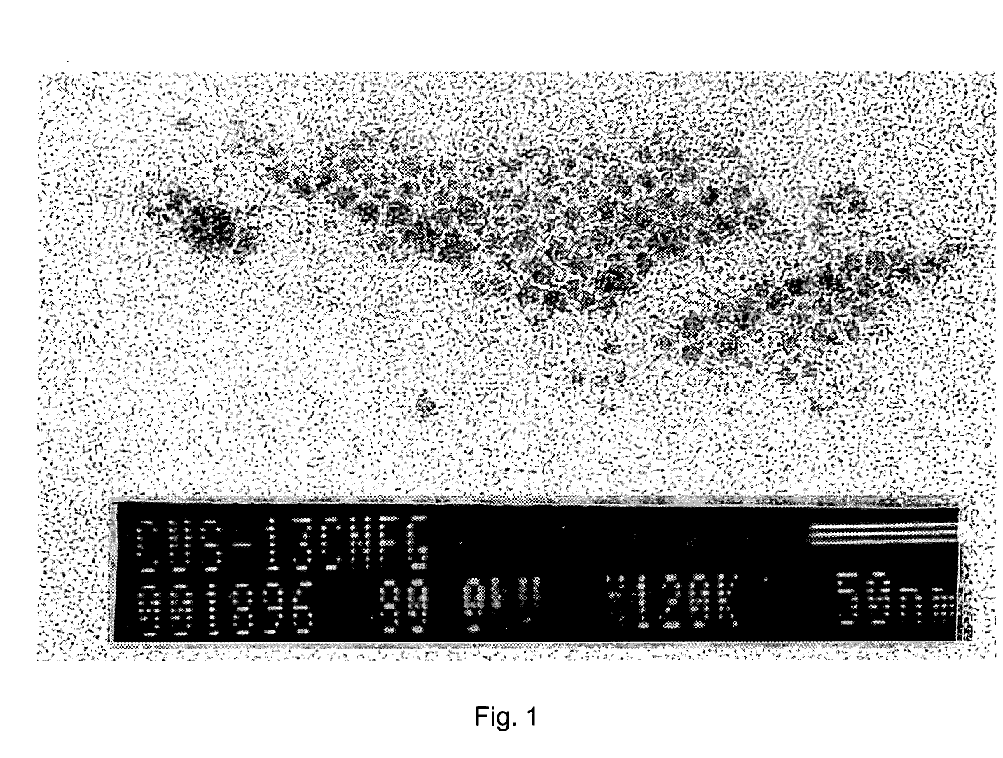

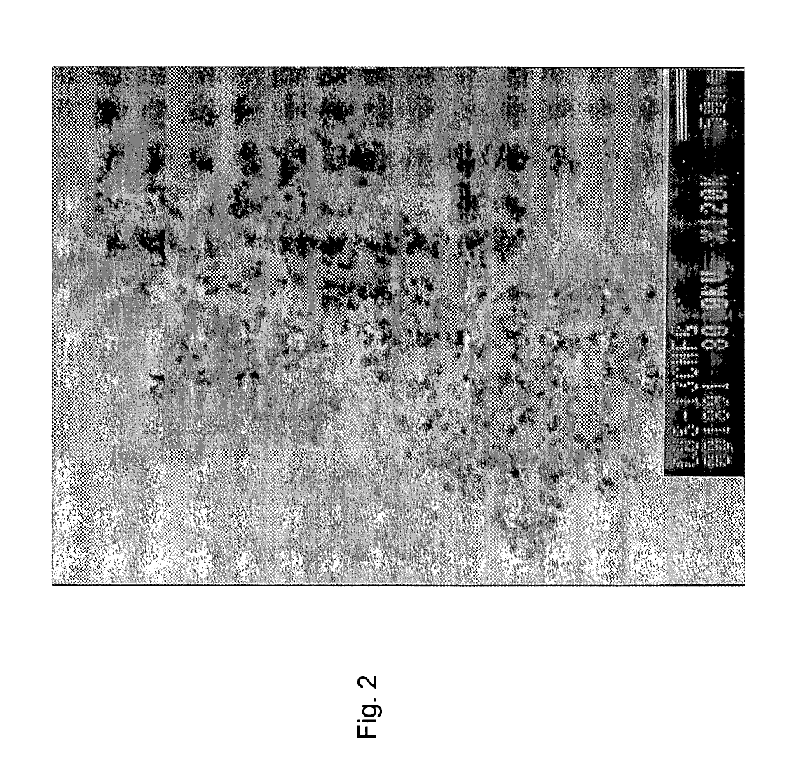

The copper dithiocarbamate complex was then decomposed in a high-boiling coordinating solvent. To prepare the nanoparticles, a solution consisting of 1.5 mmol of Cu(S₂CNEt₂)₂ in 11.0 mmol of tri-n-octyl-phosphine sulfide (TOP) was injected at 250° C. into a 10.0 mmol solution of tri-n-octyl-phosphine oxide (TOPO) or tri-n-octyl-phosphine sulfide (TOPS). All reactions were carried out in a non-oxidizing Ar environment. The progress of the reaction was monitored by extracting small aliquots (about 0.15 mL) from the reaction flask at particular time intervals and quenched in toluene. The prepared nanoparticles were stable and dispersed in a clear sample in toluene over a period of months. After evaporation, the dried powder containing nanoparticles was analyzed, by X-ray powder diffractometry to confirm the Cu_{1.8}S digenite phase and transmission electron microscopy to determine the size and shape of the formed nanoparticles. FIGS. 1 and 2 illustrate TEM characterization of Cu_{1.8}S respectively showing highly dispersed nanoparticles between 6-13 nm, with an average size of 5-8 nm.

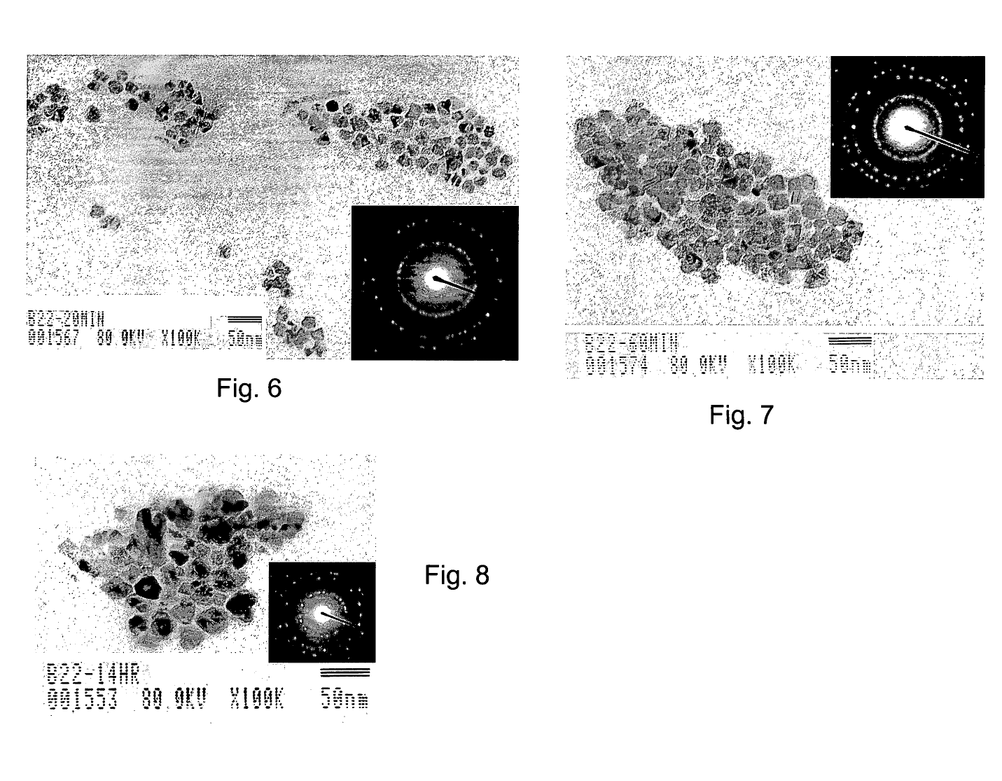

CuInSe₂ nanoparticles were synthesized by sonicating selenium to dissolve it in TOP and form a TOPSe solution. TOPO was heated to 100° C. and then purged with argon. An equimolar complex of CuAc (copper acetate) and InAc₃ (indium acetate) was dissolved in TOP. The starting material Cu:In:Se had a molar ratio of 1:1:2. The reaction took place inside a CEM microwave reaction system with electromagnetic stirring. More specifically, the Cu and In precursor solution was injected into hot TOPO and maintained at 100° C. for 1 hr, then further heated to 250° C. TOPSe was then injected and the reaction kept at 250° C. for 24 hours. The reaction mixture was cooled to about 70° C. and copious methanol was added to flocculate the nanoparticles. The final particles were separated by centrifugation and washed with methanol to remove excess TOPO and other inorganic byproduct. The powder obtained was measured by X-ray diffraction (XRD) or redissolved in hexane for TEM or optical measurements. FIGS. 1-6 illustrate the progress of the reaction, monitored as above by extracting small aliquots. The above reactions were run at ambient pressures. These reactions are amenable to batch processes, a semibatch processes or a continuous flow processes. Both the single source and multiple source processes took place in a non-oxidizing environment, such as an argon or nitrogen atmosphere.

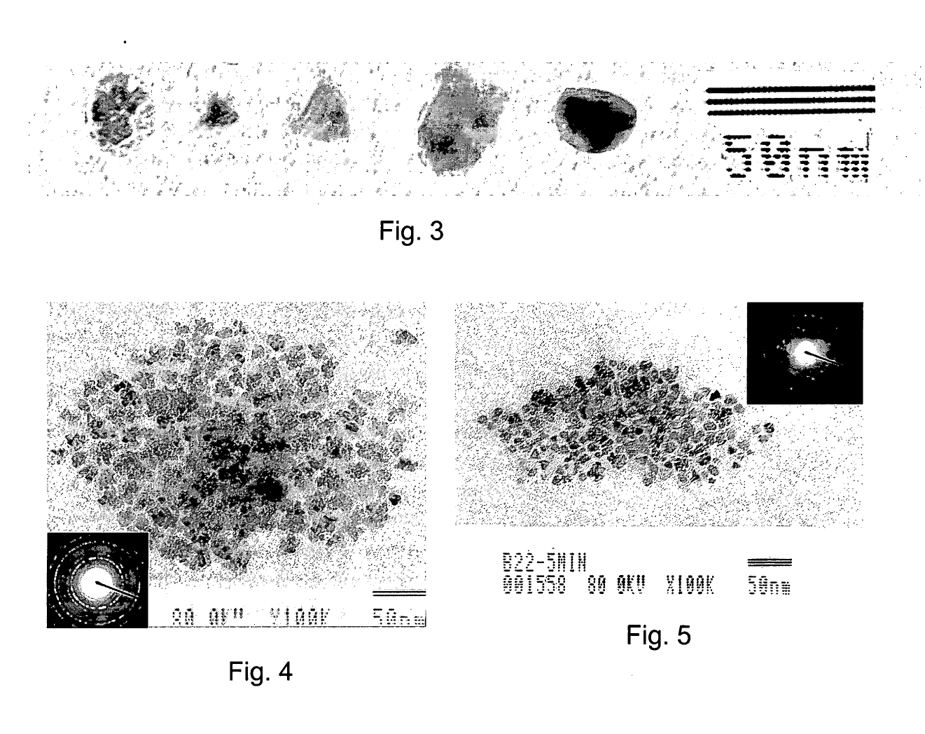

FIG. 3 shows the size and shape evolution of the CuInSe₂ nanoparticles at 1 min, 5 min, 20 min, 60 min, and 14 hrs from left to right. TEM images, with insets containing Selected Area Electron Diffraction (SAED) patterns, of the CuInSe₂ nanoparticles at these respective times are shown in FIGS. 4-8. As illustrated, the nanoparticles are polycrystalline and highly dispersed in each case and the average size is in the range of 10-30 nm.

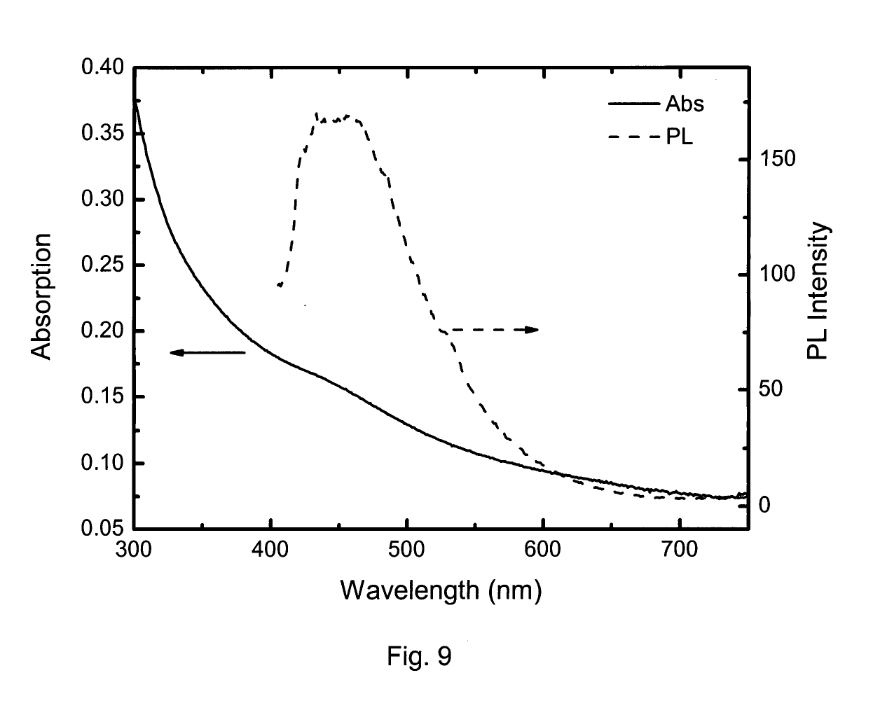

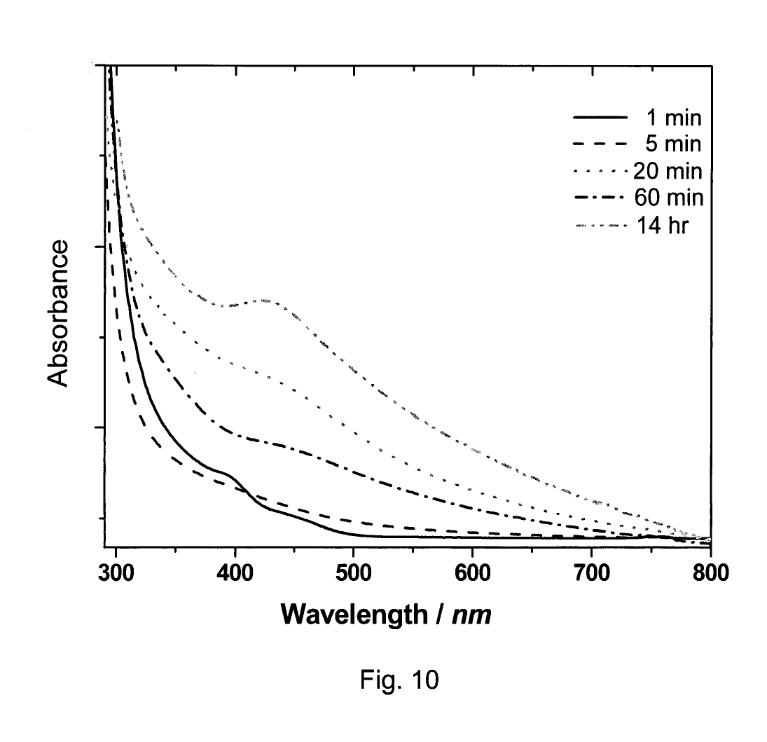

FIG. 9 illustrates the UV-visible absorption and Photoluminescence (PL) spectra of the CuInSe₂ quantum dots. There is an absorption shoulder centered at 450 nm which is blue shifted in comparison to the bulk band gap (1.1 eV) of the CuInSe₂. A broad PL peak is observed from 430 to 470 nm. FIG. 10 illustrates the UV-visible absorption of CuInSe₂ at different reaction times: 1 min, 5 min, 20 min, 60 min, and 14 hr. There is slight blue-shift of the absorption maximum from 449 to 428 nm as reaction times proceeds, which is the result of quantum confinement.

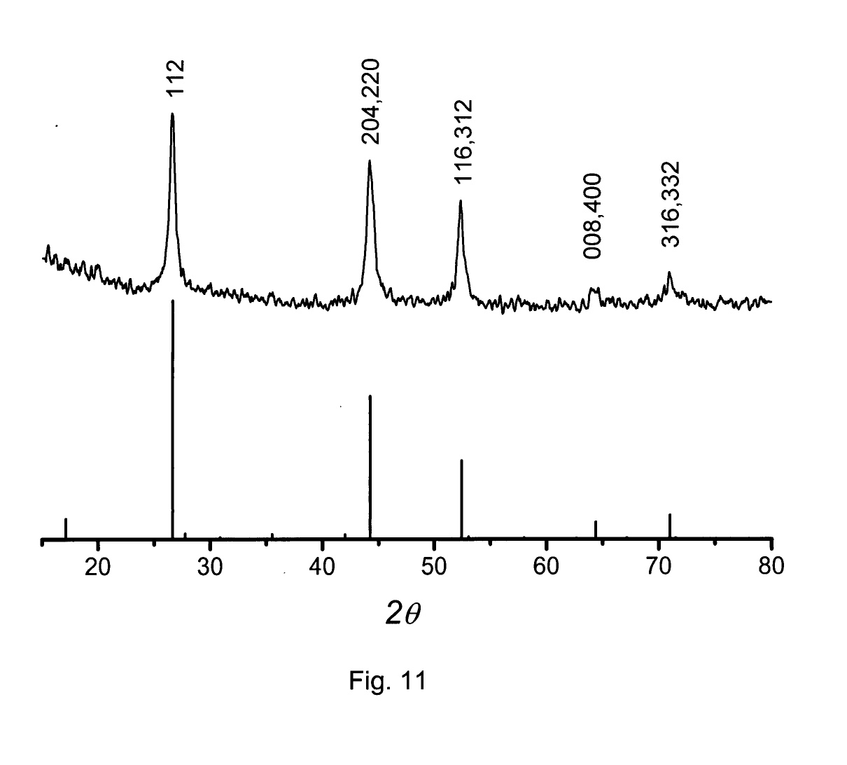

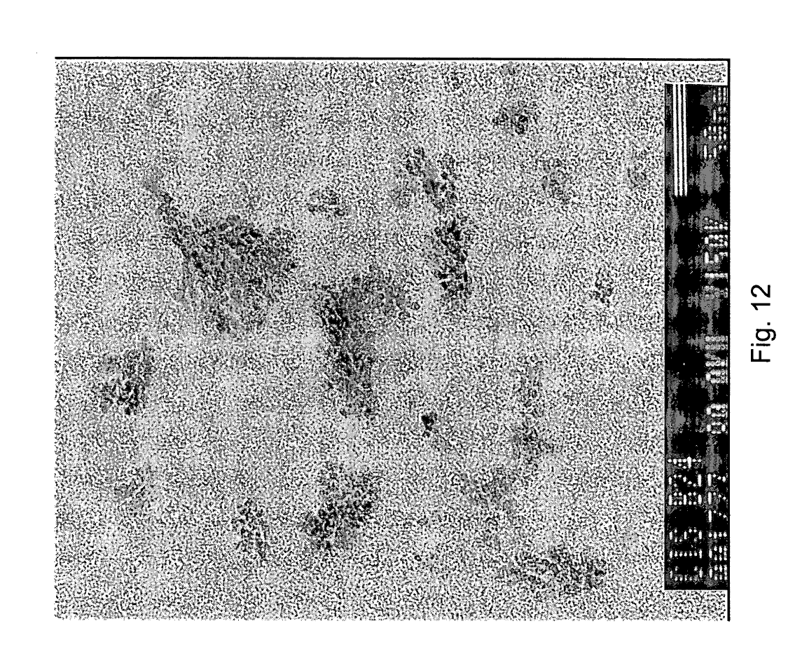

FIG. 11 shows the X-ray diffraction pattern of CuInSe₂ in which the upper pattern shows the X-ray diffraction data and the lower pattern shows the Joint Committee of Powder Diffraction (JCPDS) pattern of 80-0535, indicating the high phase purity of tetragonal CuInSe₂.

In another example, in which CuInS₂ nanoparticles were synthesized, sulfur was dissolved in TOP to form a TOPS solution. TOPO was heated to 100° C. and then purged with argon. An equimolar complex of CuAc and InAc₃ was dissolved in TOP. The starting material Cu:In:S had a molar ratio of 1:1:2. More specifically, the Cu and In precursor solution was injected into hot TOPO and maintained inside a CEM microwave reaction system at 275° C. for 30 minute with electromagnetic stirring. The reaction mixture was cooled to about 70° C. and copious methanol was added to flocculate the nanoparticles. The final particles were separated by centrifugation and washed with methanol to remove excess TOPO and other inorganic byproduct. The powder obtained were measured by X-ray diffraction (XRD) or redissolved in hexane for TEM or optical measurements. FIG. 12 illustrates the TEM images of the CuInS₂ particles. As shown in this figure, the particles were highly dispersed and the average size was in the range of 3-8 nm.

Similar techniques can be used to create other binary, ternary or quaternary nanoparticles or nanoparticles containing more elements. As above, other examples of binary and ternary nanoparticles such as CuSe, CuTe, CuInTe₂, CuAlS₂, or CuAsS₂ may be manufactured using the single or multiple source precursor method. Aluminum may be preferable as it is non-toxic and relatively cheap. Similarly, other group I metals such as Ag or Au can replace Cu in the single or multiple source precursor method by replacing the copper precursor with the desired metal precursor.

In each of the above examples using the multiple source method, a metal acetate such as a copper acetate was used as a precursor. The metal acetate and a surfactant such as TOPS were mixed together. The surfactant may reduce surface defects of the nanoparticles. An organic solvent such as hexane or tolulene, which has a higher boiling point than hexane was added to the acetate-surfactant complex. Each of the mixtures above was constantly stirred to form uniform solutions. The complex was then decomposed by heating in the solvent for minutes to hours, depending on the precursors. The progress of the reaction was monitored by extracting small aliquots from the reaction flask at particular time intervals and quenched in toluene. The solution was then cooled. As defined herein, cooling includes both actively cooling the solution, such as by quenching the solution, or passively allowing the solution to cool to a particular temperature. Evaporation of the solution produced a powder containing the nanoparticles. The nanoparticles prepared in this manner were stable over a period of months.

Heating the solution at higher temperatures leads to nanoparticles with fewer defects, i.e. better crystallinity to be obtained. This may be preferable for photovoltaic applications as the carriers (electrons and holes) created by light that is absorbed by the nanoparticles have longer free-carrier lifetimes, which results in a higher current being produced due to fewer carriers recombining at the defects. In addition, brighter and sharper band-edge luminescence (luminescence from electrons in the conduction band and holes in the valence band) is obtained, as exhibited below. In one embodiment, the solution is heated to temperatures close to the boiling point of the solvent (e.g. within about 10-20° C.), which provides nanoparticles having a high crystallinity, i.e. that are substantially defect free.

Heating the solution at lower temperatures increases the number of defects and accordingly provides a relatively short free-carrier lifetime, i.e. a short time to trap excited electrons and holes at the defects. This may be preferable in the creation of photocatalysts or applications in which defect-mediated recombination provides luminescence. Changing the heating temperature also alters the size of the nanoparticles created. The size of the nanoparticles can be accurately controlled, in various embodiments, to between about 1-100 nm, 5-50 nm, or more typically 5-10 nm.

In addition to modifying the heating temperature, changing the pressure, type or amount or ratio of precursors, concentration of capping agent, dopant, or any combination thereof permits control of the size of nanoparticles within desired ranges of sizes. For example, nitrates chlorides, or acetates may be used as precursors without substantially changing the process conditions. The amount of precursors can be varied from about 10⁻³ to 10⁻⁶ concentration in solution. The ratio between precursors in the multiple precursor method can vary between about 1:1 and 1:2 (additional metal precursor) for binary nanoparticles, and about 1:1:2 to 1:2:2 (in which the group III element varies) for ternary nanoparticles. The crystalline properties can also be affected by varying the heating rate or cooling rate, for example cooling can occur gently by simply removing the sample from the heat or suddenly by quenching, or varying the mixing rate from slow mixing by dripping material together in a minute or so to suddenly injecting the materials together within the space of a second.

The amount of material produced by the single or multiple source method may be scaled up to industrial size batches from experimental size batches. In the multiple source method, for example, the metal acetate may be obtained from any commercially available source, unlike the single source method in which the single precursor is produced first and then the nanoparticles manufactured. Thus, although multiple precursors are used, the number of manufacturing steps is decreased as the precursors are already prepared.

Many of the various methods that have been developed for synthesizing nanoparticles result in nanocrystals with a tendency to aggregate. However, highly dispersed nanoparticles may be obtained by the methods described above. Highly dispersed nanoparticles have a separation between nanoparticles on order of about 100 nm or larger in solution. A solution, as defined herein, includes any material capable of retaining the nanoparticles, such as paint or a polymer. As the characteristics of the nanoparticles change with agglomeration of the nanoparticles, the relatively large average distance between the nanoparticles causes only a small variation in the characteristics if the average distance varies slightly. In other embodiments, the average separation can be controlled to be greater than about 10 nm, greater than about 50 nm or greater than about 200 nm, or can range from about 10 nm to 1000 nm, from about 50 nm to 200 nm, or from about 100 nm to about 200 nm, for example.

In general, such dispersed nanoparticles have optical and electronic properties that are suitable for a variety of applications such as solar cells and other photovoltaics, semiconductor applications, and molecular imaging (such as biomedical imaging) or therapeutic applications such as photodynamic therapy (PDT). The use of powdery material containing nanoparticles, for instance, allows flexible solar cells to be fabricated. Non-toxic materials permit the quantum dots (QDs) to be used in diverse applications in outdoor environments. In particular, the dispersed nanocrystals have significant absorption and luminescence in the visible region, making them good candidates for visible-light applications. Dispersed nanoparticles may increase the stability and/or provide improved optical properties in the application.

Materials other than TOP, TOPO, TOPS, or TOPSe, can be used to cap the molecules forming the nanoparticles. That is, in general the ends of the nanoparticles are non-polar molecules. By introducing different precursors, a ligand exchange can be produced, which permits the nanoparticles to be “capped” or the molecules at the end of the nanoparticles controlled. For example, polar molecules such as NH₃ may replace the non-polar molecules normally disposed on the ends of the nanoparticles. This permits the creation of charged nanoparticles, which are soluble in water and/or pass through cell membranes. Other capping agents, such as phospholipids or soap-type molecules may be used to cap the nanoparticles. These capping agents perform various functions such as protecting the nanoparticles from oxidation, dispersing the nanoparticles in solvents such as organic or fluorocarbon solvents, and/or control aggregation of the nanoparticles.

The capping agent may bond in any fashion (ionic, covalent, dipolar, dative, quadrupolar or van der Walls interactions) to the nanoparticles. Capping may occur through a combination of organic ligands and inorganic small molecules. Capping agents include compounds having the general formula (R)ₙ−X, where X is an atom or functional group. Each R group may be hydrogen, an aryl group or an alkyl group. X may be an atom that includes nitrogen, carbon, oxygen, sulfur, and phosphorus or a functional group that includes a carboxylate, a sulfonate, an amide, an alkene, an amine, an alcohol, a hydroxyl, a thioether, a phosphate, an alkyne, an ether, an ammonium group or combinations thereof. Each group may be independent from each other.

In other embodiments, the nanoparticles may include a metallic and/or non-metallic dopant. The non-metallic dopant may be selected from the group consisting of boron, carbon, silicon, germanium, nitrogen, phosphorous, arsenic, sulfur, selenium, tellurium, fluorine, chlorine, bromine, iodine, and combinations thereof. The metallic dopant can be, for example, a transition metal. In some embodiments, the nanoparticles contain from about 0.05 to about 20 percent dopant. In some embodiments, the nanoparticles contain from about 0.1 percent to about 15 percent dopant. In some embodiments, the nanoparticles contain from about 0.5 percent to about 12 percent dopant. In some embodiments, the nanoparticles contain from about 1 percent to about 10 percent dopant. In some embodiments, the nanoparticles contain from about 4 percent to about 8 percent dopant.

Use of nanoparticles in various applications depends on the optical and electronic characteristic of the nanoparticles. The optical properties are dependent on the shape, size and dispersion, as well as the composition of the nanoparticles. The nanoparticles grow and change shape with time and are dependent on the precursors used.

The various nanoparticles described herein absorb light in the visible region of the electromagnetic spectrum (i.e., light having a wavelength ranging from about 380 nm to about 800 nm) and/or near infrared region (from about 800 nm to about 2 μm). The wavelength of light absorbed by the nanoparticles varies with the size of the nanoparticle and the concentration of dopant in the nanoparticle. In general, larger particles absorb at longer wavelengths than smaller particles. In addition, lower concentrations of dopant correlate with absorption at shorter wavelengths while higher concentrations of dopant correlate with absorption at longer wavelengths.

The nanoparticles produced by the above methods return about 25% or more of the energy incident thereon as luminescence. The amount of luminescence from the nanoparticles is much greater than that obtained with conventional organic dyes. In fact, the amount of luminescence and wide variation in wavelengths available due to differences in the size, shape and/or composition of the nanoparticles permit the use of the nanoparticles in an assortment of uses.

In one set of experiments, the absorbance and fluorescence spectra of copper-based nanoparticles were measured on a fluorescence spectrophotometer. X-ray analysis of the samples was carried out using a Philips PW3710 X-ray powder diffractometer. Femtosecond time-resolved transient absorption measurements were recorded on a laser pump-probe system consisting of an amplified erbium-doped fiber laser, which is frequency doubled to 780 nm and amplified in a regenerative amplifier. The femtosecond laser produced pulses with 120 fs full width-half maximum duration and 800 μJ output energy per pulse at a repetition rate of 1 kHz. A small portion of the fundamental output pulse train was used to generate white light in a 2 mm sapphire crystal, the remaining laser light was used to either frequency double or triple the fundamental to achieve 390 or 260 nm, respectively. To obtain different excitation wavelengths, an optical parametric amplifier (OPA) was employed to facilitate the sum frequency mixing and doubling of the signal or idler. Likewise, the probe wavelength range was extended beyond the white light spectrum by using an OPA, which includes difference frequency mixing techniques for probing in the mid-IR range. The pump-probe experiments were all carried out at ambient temperature. For the femtosecond laser spectroscopy measurements, the excitation beam was modulated by a chopper with a 100 Hz frequency. The probe light was used with reflective optics to avoid white light dispersion. Measurements were conducted with the excitation beam focused to a spot diameter of about 500 μm and the probe beam to 100 μm. The nanoparticle solution was placed in a 2 mm path length quartz cuvette and continuously stirred by a cell stirrer to avoid permanent bleaching of the pump-probe volume element in the solution. Matrixes of wavelength versus delay time were determined and analyzed by a single-value decomposition method.

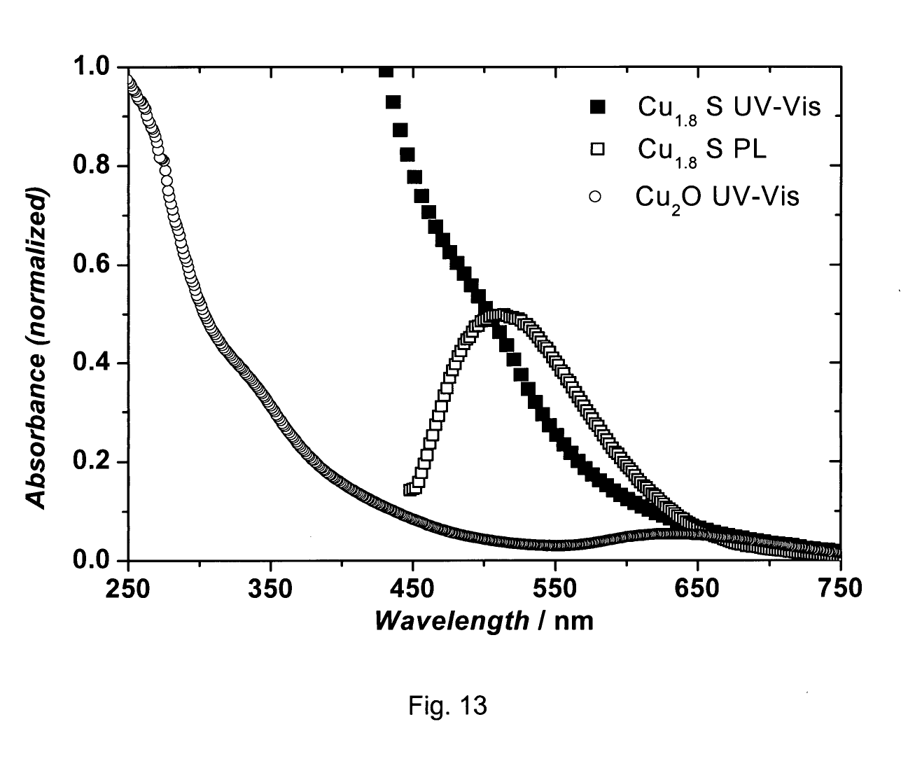

FIG. 13 shows room temperature UV-visible absorption and PL spectra of synthesized Cu_{1.8}S (˜20 nm) and Cu₂O (6.4 nm) nanoparticles in a hexane solvent. The absorption spectrum for the Cu_{1.8}S nanoparticles shows a band gap absorption centered at 510 nm. A blue-shift of band gap energy by 0.9 eV relative to bulk digenite copper sulfide is the direct result of the spatial confinement or quantum confinement (the smaller the particle size, the larger the band gap energy, and the more blue-shift of the absorption peak). The photoluminescence spectrum of Cu_{1.8}S nanoparticles shows an emission maximum around 515 nm, which has lower energy than the absorption maximum. This indicates that the absorption occurs due to excitation into intrinsic electronic states, while the emission occurs from trapping sites. In comparison, the absorption spectrum of Cu₂O nanoparticles shows two hump-like optical absorption features at about 260 nm and 340 nm, which are attributed to band-to-band transition in the crystalline Cu₂O. Another broad feature centered at 630 nm is also observed, which is probably due to the forbidden band gap transition. There is no observable emission spectrum for the as synthesized Cu₂O nanoparticles, which is due to the forbidden band gap transition and fast non-radiative decay.

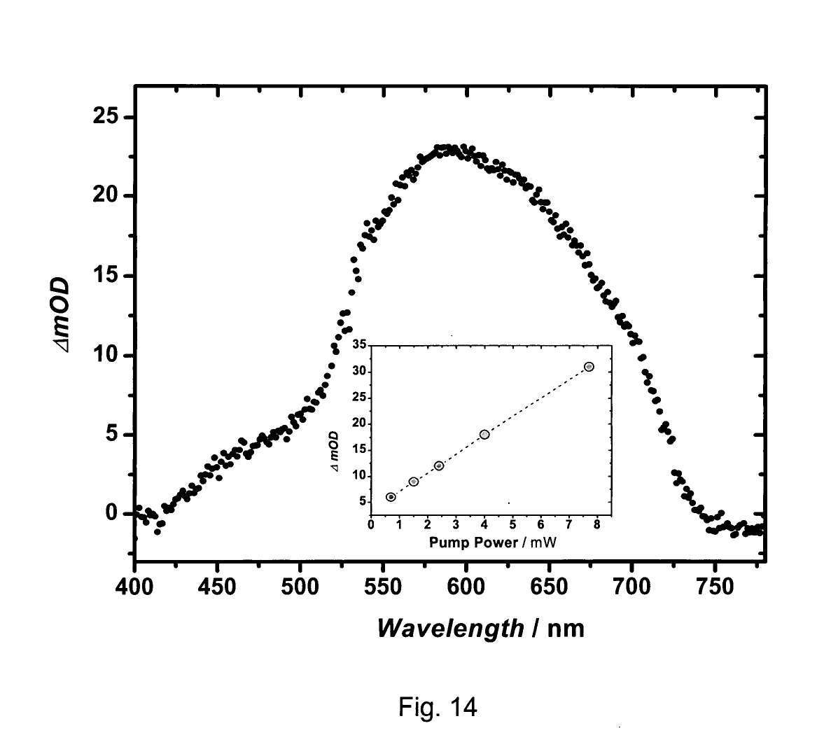

Upon femtosecond laser excitation at 390 nm of the Cu_{1.8}S nanoparticles, a strong excited state absorption (a positive transient signal) from 450 to 750 nm with a maximum around 600 nm was observed, as shown in FIG. 14. No saturation was observed up to 22 excitations per nanoparticles. The transient absorption band is almost symmetric except a small dent between 450 and 550 nm. The overall peak intensity was excitation power dependent as seen in the inset FIG. 14. The linearity of the absorption intensity with no saturation happening indicates that the number of surface traps exceed the number of excited carriers. The dent in the transient absorption spectrum around 510 nm is found to be the superposition of transient absorption and transient bleach in this range, as evidenced by the lowest energy transition around 510 nm. Transient bleach is the direct result of excited-state filling, which leads to a partial saturation of the band gap transition as long as the excited state are not fully populated. The combination of transient bleach and transient absorption around 510 nm should give a more complex relaxation dynamics compared with dynamics at 600 nm (intraband gap) range.

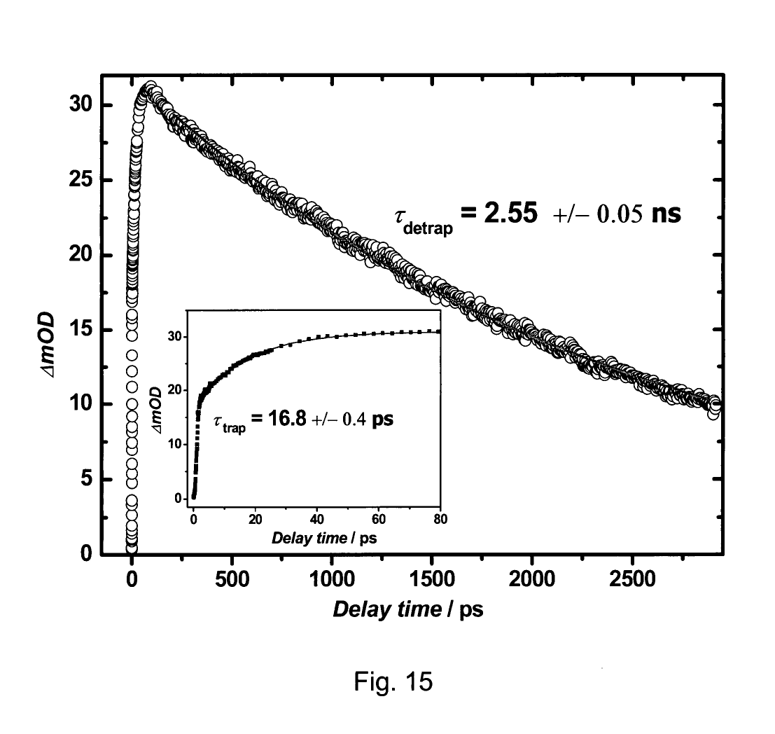

The relaxation of the transient absorption at 600 nm has been fitted to a single exponential decay and the lifetime is found to be 2.5 ns, as shown in FIG. 15. The relatively long lifetime indicates that the monitored transient absorption is from the trapped charge carriers. The trapping process should be possible to observe when we look into the rising part of the kinetic trace as long as the trapping is not faster than the experimental time resolution of 120 fs. The inset in FIG. 15 shows the trapping under laser excitation. Two trapping components are present: a faster increase in absorption within the first 400 fs and a slower component with a lifetime of 16.8 ps. The fast component is probably due to excited-state absorption of the delocalized hot carriers or fast carrier trapping on a subpicosecond time scale. The slower component can be assigned to the trapping process of carriers and the concomitant depopulation of the band edge.

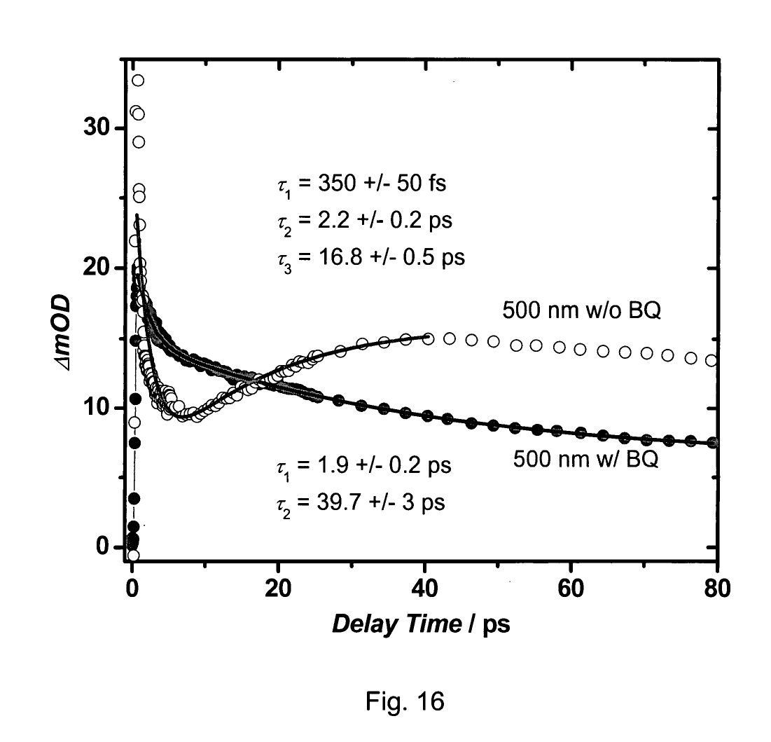

Using the technique of fast electron removal via ultrafast electron transfer across the nanoparticles interface, it is possible to assign the observed transient signal to either hot electron or hot hole. The adsorbed benzoquinone then acts as an electron acceptor to remove the hot electrons in less than the laser pulse duration (120 fs). Then the detailed electron or hot dynamics can be deduced from measurement with and without benzoquinone. Due to the combination of transient absorption and transient bleach, the kinetics monitored at 500 nm, shown in FIG. 16, is more complex than the one observed at 600 nm. The relaxation dynamics at 500 nm without any benzoquinone revealed three components in short time scale, a faster 350 fs decay, a slower 2.2 ps decay and a 16 ps rise. The 16 ps rise can be assigned to the dynamics of the electron trapping process which is consistent with the dynamics shown at 600 nm. With the electron removal by benzoquinone, the relaxation dynamics from the excited electrons will be quenched. The quenched 350 fs component can be assigned to the intraband relaxation of the hot electron while the 2.2 ps component remains unquenched and can be assigned to the relaxation of the hot hole. There is a new decay component of 39.7 ps when benzoquinone is absorbed on nanoparticles surface that may be due to the trapping of the hole or the recombination of the carriers.

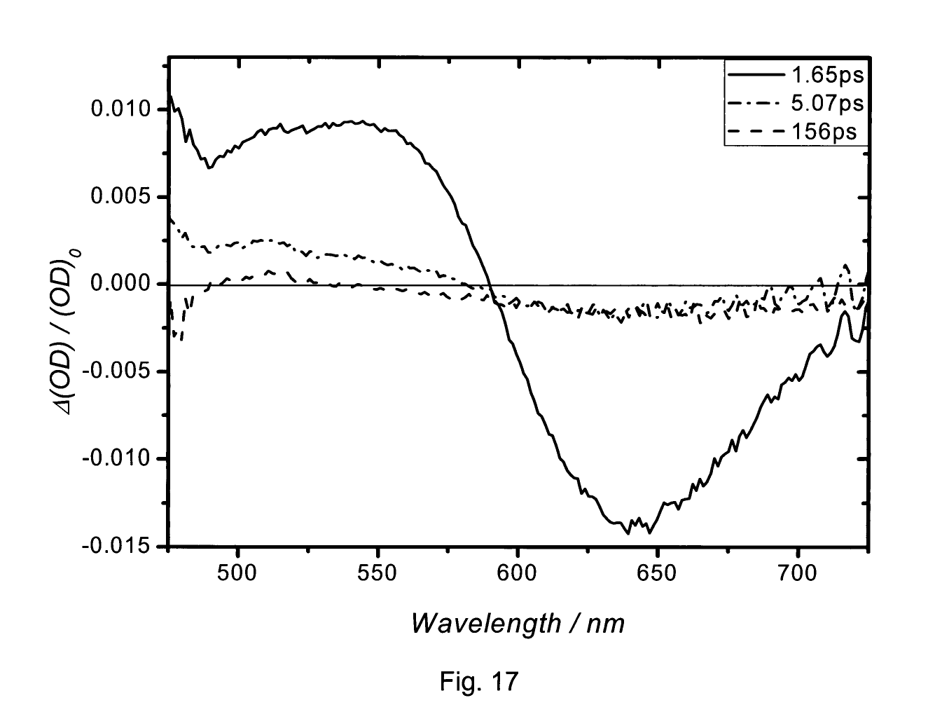

Transient absorption spectra of 6.4 nm Cu₂O nanoparticles at 1.6 ps, 5.0 ps, and 160 ps are shown in FIG. 17. Upon 390 nm femtosecond laser pulse excitation, two main features were observed. A strong excited state absorption was observed from 475 nm to 590 nm, while a strong bleach signal was observed from 590 nm to 725 nm. The maximum of the initial transient bleach was centered at 640 nm while the steady state absorption is centered at 550 nm.

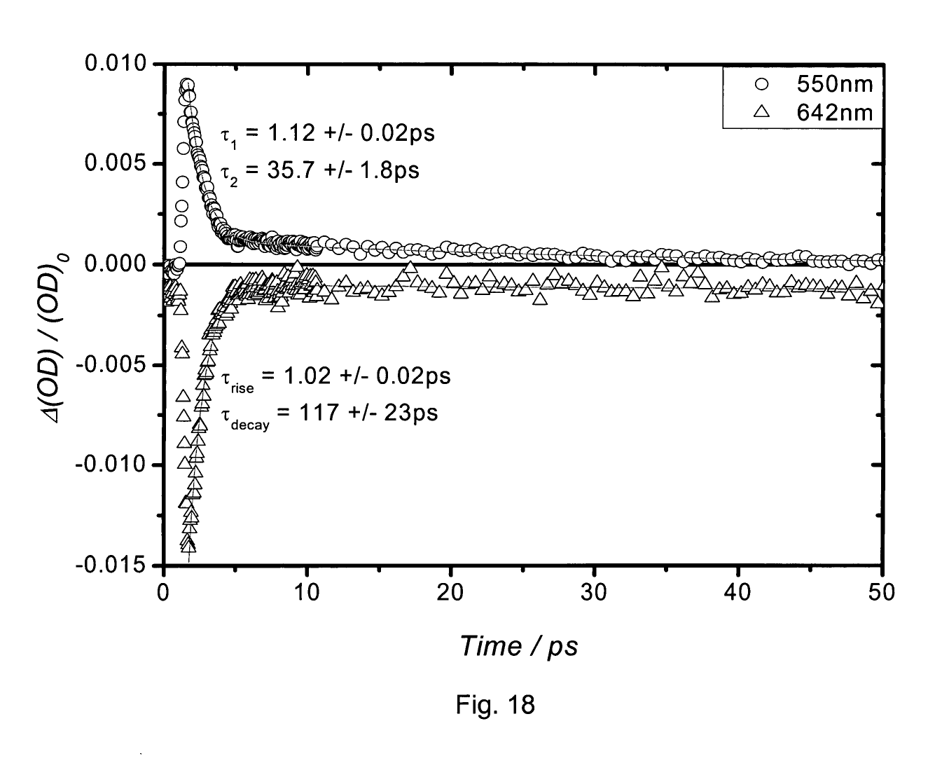

The decay dynamics of the laser pulse induced transient absorption monitored for the 6.4 nm Cu₂O quantum dots at 550 nm was fitted as bi-exponential decay, as shown in FIG. 18. A lifetime of 1.1 ps was measured, corresponding to a short-lived trap state population. The longer lifetime of 35.7 ps corresponds to longer lived trap states. The rise time of laser induced bleach, monitored at 642 nm, was 1.02 ps, which was attributed to the population of the lowest excitonic state. The decay time of 117 ps depicts a shorter lifetime component of the exciton. There is a slight delay of 0.16 ps to reach the bleach maximum corresponding to the transient absorption maximum, which can be explained with surface state population from initially excited states. Trap states are populated before the exciton is formed in its ground state. A very long lived transient bleach component was observed in the nanosecond time regime, which was confirmed by nanosecond flash photolysis. It is the lifetime of the excitons at the band edges. The enormously extended exciton lifetimes are due to the forbidden band gap transition at the Γ point.

To build efficient photovoltaic or other semiconductor devices, long electron-hole mobility lifetimes may be desirable. Several effects of particle size changes are taken into account. First, larger nanoparticles show a decreased surface-to-volume ratio, which may be favorable if it reduces the relative number of surface trapping sites per atom in a quantum dot. In addition, surface traps may be shallower for larger nanoparticles since lattice strain reduces as the nanoparticles grow. This may lead to longer mobility lifetimes due to slower trapping rates in larger nanoparticles. Shallower trapping sites may also lead to shorter lifetimes within a specific trap and the ability to repopulate the conduction band thermally. Second, smaller nanoparticles may have a smaller electron-phonon coupling due to the reduced density of states, which may translate into longer intrinsic lifetimes and slower internal relaxation for small nanoparticles. Based on these opposite trends, an intermediate size range in which a compromise between trap concentration and trap depth (favoring large nanoparticles) and reduced electron-phonon coupling (favoring small nanoparticles) exists. A series of Cu_{1.8}S nanoparticles of different sizes were prepared and investigated for their electron relaxation dynamics.

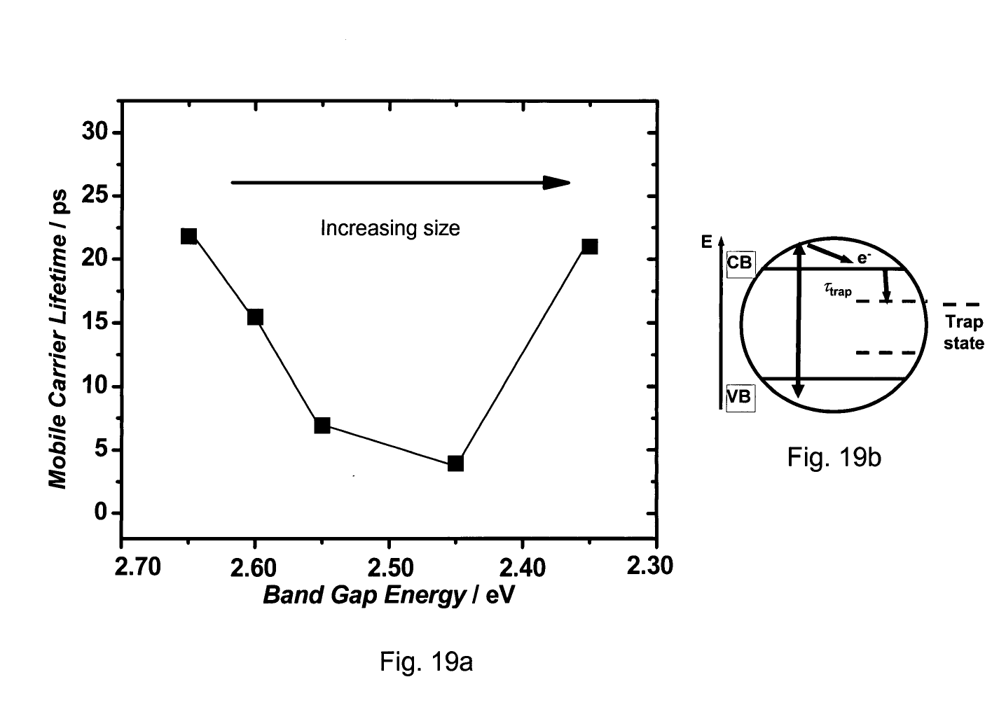

The relaxation times for copper sulfide nanoparticles, as measured in the femtosecond pump-probe experiment, suggest a correlation between particle size and the trapping behavior. The electron trapping times for Cu_{1.8}S quantum dots of different sizes for band gap energies obtained from TEM and uv-visible absorption spectroscopy are shown in FIG. 19a. FIG. 19b is a diagram of hot electron trapping. The short lifetime component (τₜᵣₐₚ, solid squares) was shown to undergo a U-shaped behavior, indicating that the longest lifetimes are achieved with either large or small particles but in the intermediate size range there is a region with short trapping times.

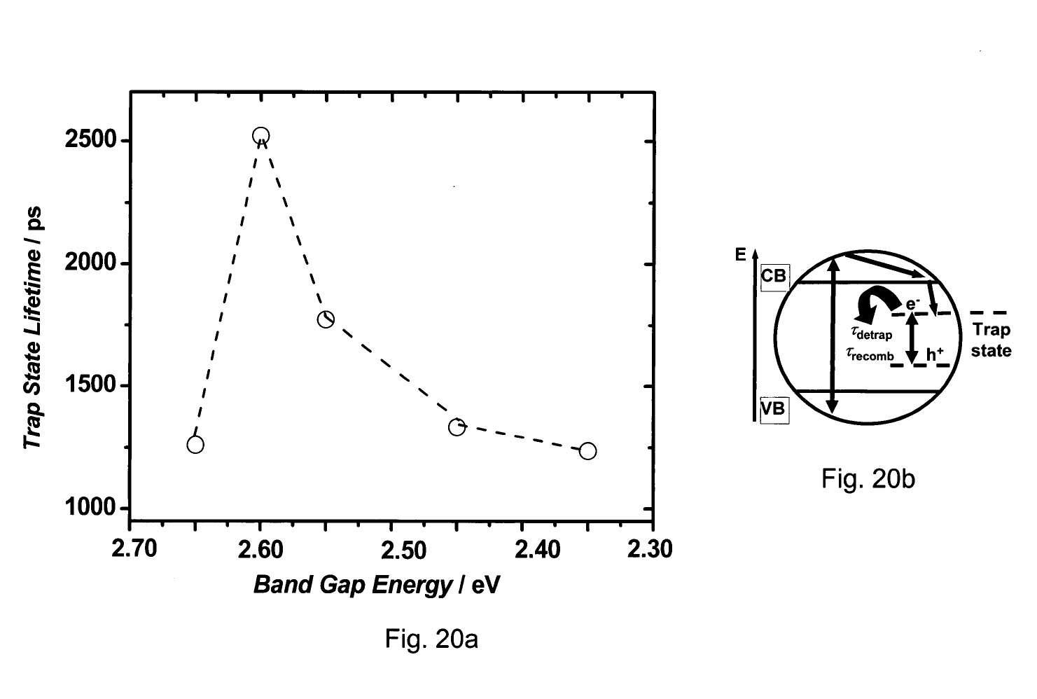

FIG. 20a illustrates the trap state lifetimes for quantum dots of various sizes with band gap energies. The longer lifetime component (τ_{recomb}, open circles) indicates that the nanosecond lifetimes of carriers in the trap states have a tendency to become shorter for larger nanoparticles, except for the very small ones where a steep increase in the trapping time is observed. FIG. 20b is a diagram of electron-hole recombination at the traps. This observation leads to the conclusion that the small and large nanoparticles seem favorable in terms of the spectral response and for creating long-lived mobile charge carriers in devices built from CuₓS nanoparticles. In contrast, the medium sized nanoparticles show the fastest trapping times and not much longer trap lifetimes.

While the carrier lifetime in the copper sulfide nanoparticles is only 3-22 ps, depending on particle size, it is much longer in the case of the copper oxide nanoparticles, which have time components of 117 ps and several nanoseconds. The differences of the carrier lifetimes may be due to varying surface structures of the two quantum dot systems. However, the copper sulfide nanoparticles exhibit similar dynamics to that of cadmium based II-VI semiconductor nanoparticles in that it shows more trapping behavior. Both copper sulfide and copper oxide nanoparticles materials show ground state absorption throughout the whole visible range. While the absorption of the copper oxide is much weaker in the mid-visible range, the weaker absorption may be compensated with the much longer excited-state lifetime that is provided by the Cu₂O nanoparticles.

Faster electron-hole recombination processes may be better for certain applications such as light emitting devices or light emitting diodes and optical switches. Coating the nanoparticles with a different wider band gap semiconductor, such as coating CuS with CdS or ZnS, may reduce the non-radiative rates. Coating the nanoparticles with a smaller band gap semiconductor like Ge may increase the photoluminescence lifetime in the particles. For certain applications, the photoluminescence lifetime may be increased through the use of different capping ligands.

Both copper sulfide and copper oxide may be used as absorber materials in solar cell assemblies, as may other of the nanoparticles. The digenite phase (Cu_{1.8}S) has the most red-shifted absorption onset of the stable CuₓS phases. Compared to Cu_{1.8}S the absorption intensity of the Cu₂O nanoparticles is weak but extends to the red edge of the visible spectrum. The Cu₂O nanoparticles have long carrier lifetimes, which may be desirable in photovoltaic devices, described below.

As mentioned above, the nanoparticles may be used in photovoltaic devices. A photovoltaic device is a p-i-n diode containing a p-type semiconductor anode, an n-type semiconductor cathode, and an intrinsic region between the anode and cathode. The p-i-n diode is sandwiched between thin optically transparent electrical contact layers such as ZnO and disposed on a glass substrate. The bandgap between the conduction band and the valence band in the semiconductors is usually between about 1.1 and 1.7 eV, which is large enough to absorb visible light (about 3.4 to 1.8 eV). Although many photovoltaics are formed from indirect bandgap semiconductors, such as silicon, for efficiency purposes, it is desirable if the semiconductors are direct bandgap semiconductors, i.e. the minimum of the conduction band and the maximum of the valence band are aligned in k-space. The intrinsic region contains the nanoparticles. The nanoparticles can be used to form a single crystalline structure (a single bandgap throughout the layer) whose bandgap is tailored to the spectrum of the light incident thereon or to form a stack of quantum wells in the intrinsic region whose absorption is tailored. The intrinsic region containing nanoparticles can increase the conversion efficiency of light incident thereon, for example, energy from the sun in solar cells, from current the 20% to a calculated value of about 66%.

Nanoparticles may be used in a number of applications other than the lightweight, flexible thin-film photovoltaics discussed above. Other examples of applications in which nanoparticles are used include, but are not limited to: light sources such as light emitting diodes (LEDs) and coherent light sources, displays, memory devices, compositions to cover surfaces, and biological applications. In some applications, nanoparticles may be poured into polymers or various aqueous media, for example, into which they disperse well.

LEDs or other light sources may be formed using nanoparticles in an ordered structure. The LED contains an anode and a cathode electrically coupled to a layer containing nanoparticles. For example, the nanoparticles may be embedded in an emission layer that is disposed between layers that transport carriers. Alternatively, a coating that includes the nanoparticles can surround an emission region. The wavelength of the light source may be independent of the applied voltage or may be dependent on the applied voltage.

Displays such as liquid crystal displays (LCDs) or other flat panel displays can use the nanoparticles to generate light. For example, nanoparticles can be used to provide light for the display in an environment in which the ambient light is not sufficient to provide adequate lighting. Thus, in one example, the display contains a pair of substrates with a layer whose optical properties change with applied electric field (such as a liquid crystal layer) therebetween, and a light source using the nanoparticles external to the substrates. The light source can contain a LED, as above. The anode and the cathode together are configured to conduct current to the nanoparticles, which produce light in response to the current. For flat panel displays, nanoparticles that emit different colors, for example, red, blue, and green, may be used in alternate cells.

A memory device (such as a flash memory, or non-volatile memory such as an electrically programmable read-only memory or electrically-erasable programmable read-only memory EEPROM) includes a source from which electrical charge originates, a drain to which the electric charge travels, a channel that separates the source and the drain, a floating gate above the channel, a control gate adjacent to the floating gate, and an oxide positioned between the control gate and the floating gate. The floating gate includes nanoparticles.

Nanoparticles can be used in a coherent light emitting device (e.g. a laser). The laser includes an excitation source through which energy is applied to the nanoparticles, an optical cavity that directs light produced by the nanoparticles, and mirrors on the ends of the cavity. When the excitation source applies energy to nanoparticles, the nanoparticles produce light, which is directed by the optical cavity. One of the mirrors reflects substantially all wavelengths other than the wavelength to be emitted by the laser and reflects up to about 99% of the wavelength to be emitted. The other mirror reflects the wavelength to be emitted by the laser.

Nanoparticles that are non-toxic or environmentally benign are desirable for biological or environmental applications. The nanoparticles may also be passivated or coated with a mixture of organic molecules to enhance their non-toxicity. Nanoparticles may be used, for example, for multimodal imaging, and may be used for molecular imaging such as biomolecular imaging. For example, the nanoparticles may be disposed in the human (or other animal) body or in tissues external to the human body as tracers, internally for imaging the human body for diagnostic purposes or for treatment of cancer using photodynamic therapy (PDT).

Nanoparticles can be used as the photosensitizing agents, to entrap water-insoluble photosensitizing agents or may effective drug carriers in aqueous media. Nanoparticles absorb strongly and, as mentioned above, the emission spectrum from nanoparticles is tunable and depends on the size, shape and composition of the nanoparticles. Since there is minimal light scattering and absorption in the near-IR region of the spectrum, light of low intensity can be used to penetrate tissue to depths of several cm, thereby allowing access to deep-seated tumors. Additionally, nanoparticles can be coated to make them water soluble and biocompatible. Thus, in one embodiment, the nanoparticles are activated at with light, energy is transferred from the activated nanoparticles to an oxygen molecule to form a reactive singlet oxygen, and the reactive singlet oxygen is reacted with a cancer cell to destroy the cancer cell.

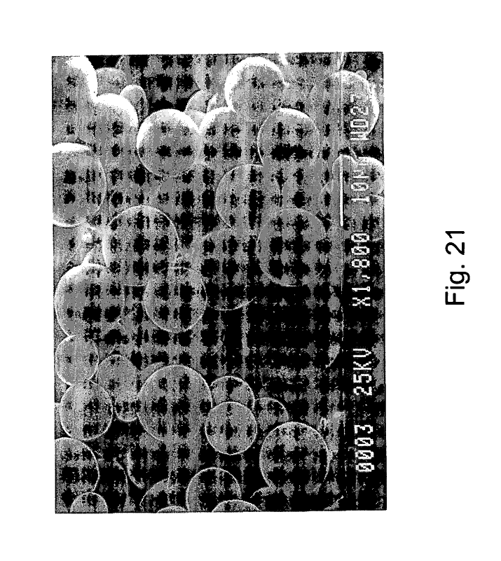

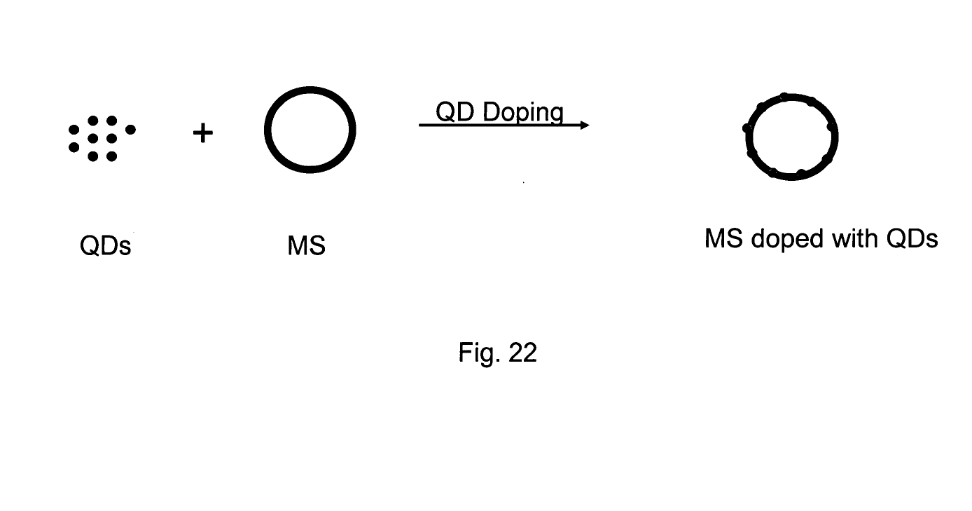

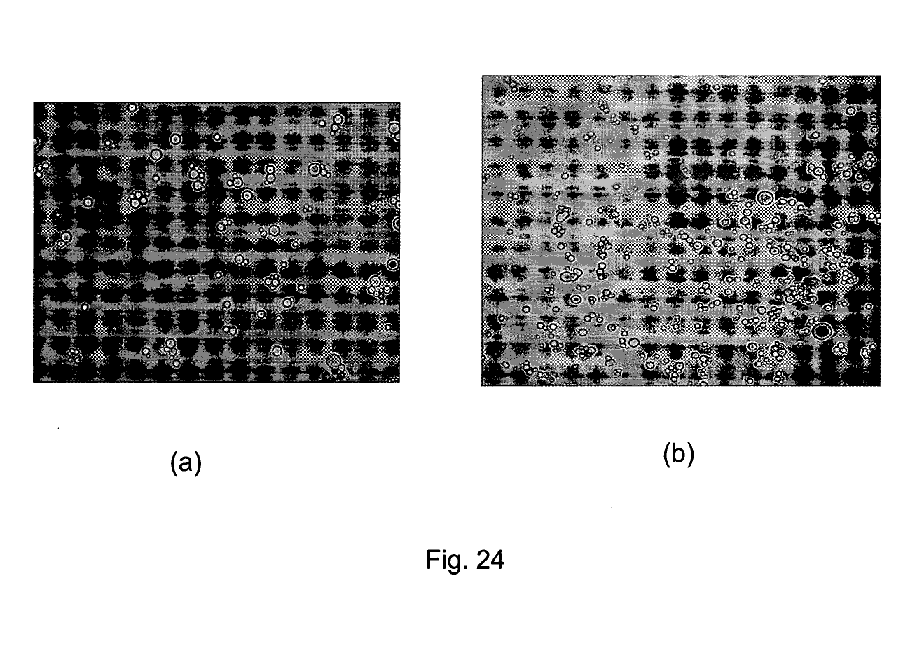

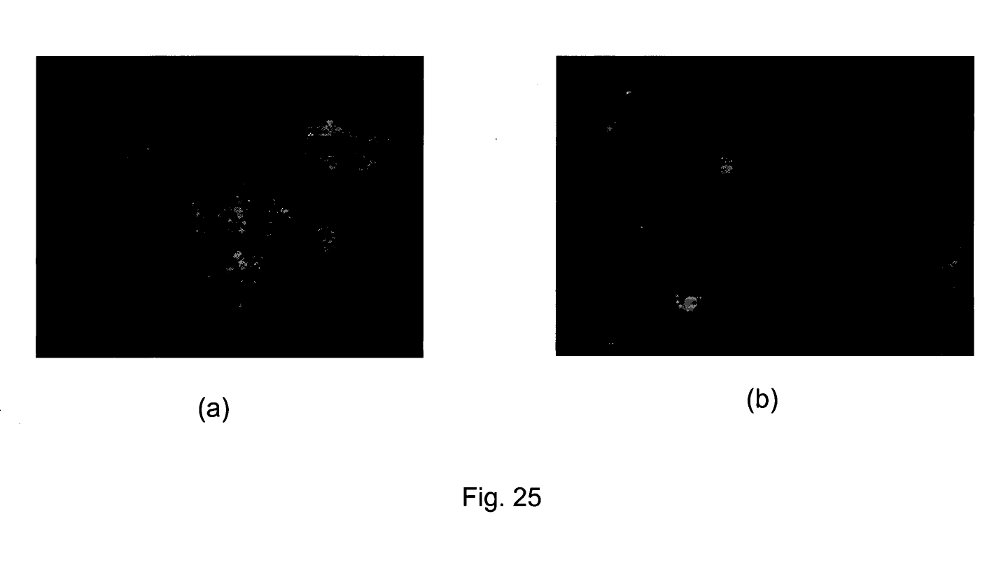

The nanoparticles can also be used to deliver drugs. In such applications, the nanoparticles (QDs) can be incorporated into spheres such as protein spheres. Protein spheres have an average size of 1 μm and are labeled MS in FIG. 22, however, as known by one of skill in the art, other types of spheres with other sizes may be used so long as they are able to incorporate nanoparticles therein. The spheres are injected into cells via ultrasonic excitation. Spheres containing the nanoparticles that are 1-5 μm in diameter are shown in FIG. 21. These nanoparticle-doped spheres are superior to conventional ultrasound contrast agents as the quantum dots can simultaneously generate ultrasonic contrast due to a difference in acoustic impedance and act in multiple therapeutic manners as drug delivers and in PDT. Optical micrographs of undoped bubbles and undoped spheres are shown in FIGS. 24(a) and (b), respectively. Fluorescence image of bubbles and spheres doped with CdTe nanoparticles are shown in FIGS. 25(a) and (b), respectively. All of the optical micrographs and fluorescence images are shown at 20X magnification.

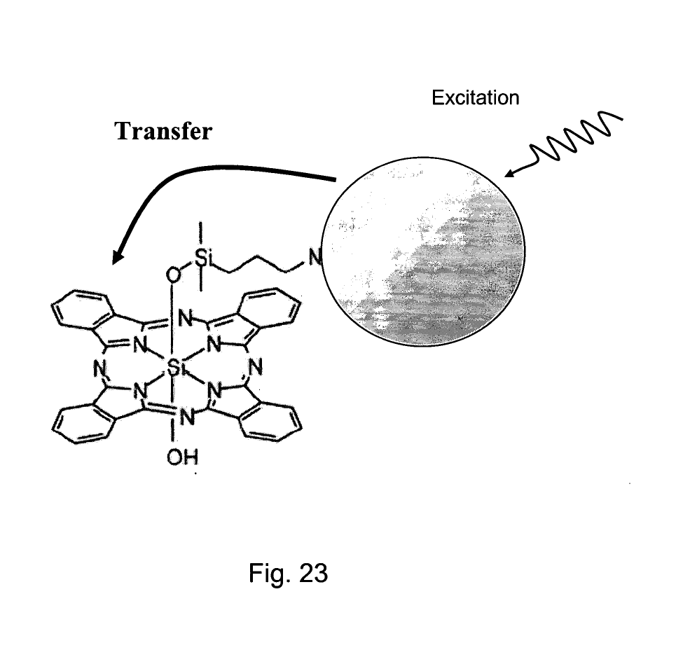

Ultrasound-mediated or other energy-mediated destruction of spheres is shown in FIG. 23. As shown, energy impinges on the nanoparticle-containing sphere. When a sufficient amount of energy is supplied to and absorbed by the protein sphere, the sphere bursts and delivers the nanoparticles to the site to react with the cell or tissue (shown as the chemical compound in FIG. 23). This method permits efficient delivery of drugs or other compounds to the cells, tissue or other area of interest in the patient. As indicated non-toxic nanoparticles are well suited to these applications. Examples of binary or ternary nanoparticles that can be used to deliver drugs through this include, but are not limited to CuₓS, CuO, CuSe, CuTe, AlS, AlSe, AlTe, CuInSe₂, CuInS₂, CuInTe₂, or CuAlS₂, or similar nanoparticles using Ag or Au. Copper sulfide nanoparticles may be especially well suited to these applications.

In fact, semiconductor nanocrystals are among the best fluorescent labels available for cellular and other imaging. Semiconductor nanocrystals exhibit narrow and symmetric emission spectrum, continuous excitation spectrum, high quantum yields and resistance to photobleaching over the conventional fluorescent dyes for biomedical applications. Furthermore, nanocrystals themselves offer, in principle, the ability to modify their surface properties for various applications, such as site-specific drug delivery and imaging.

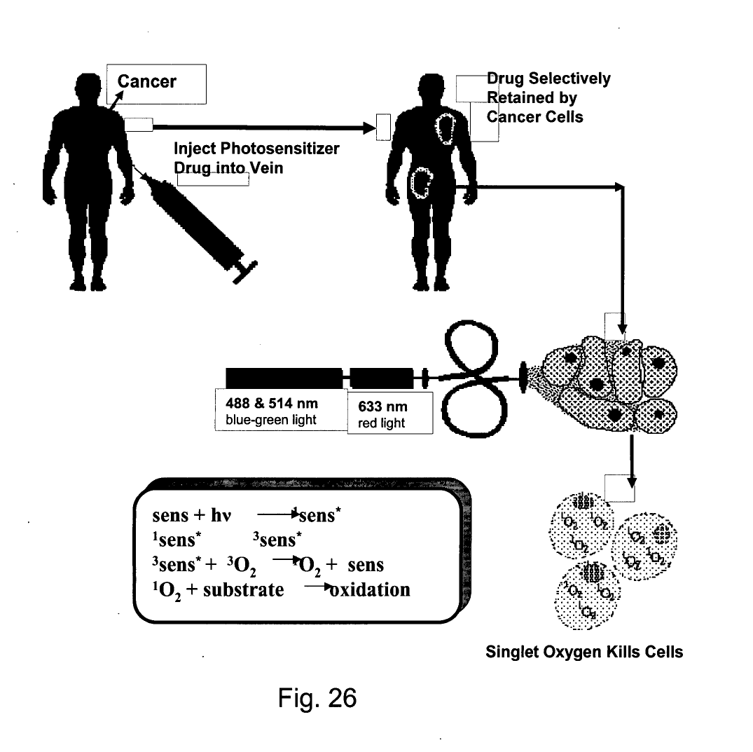

FIG. 26 illustrates a method of applying nanoparticles internally to the human body in PDT to initiate apoptosis of cancer cells. In FIG. 26, a photosensitizer agent or drug (sens in the equations) is prepared and injected into a human suffering from cancer. The drug is selectively retained by the cancerous cells and tissues. The photosensitizing agent becomes activated by light (hv in the equations), for example, visible light such as blue light at 488 nm, green light at 514 nm and/or red light at 633 nm. This light does not react directly with the cancerous cells and tissues. The photosensitizer, initially in a singlet excited state (¹sens^{*}), is exposed to the light and gains enough energy to be converted into a triplet excited state (³sens^{*}). The photosensitizing agent in the triplet excited state transfers its energy to nearby oxygen molecules, which are in a triplet state (³O₂), and reverts back to its unexcited state (sens). The oxygen changes into a reactive singlet oxygen state (¹O₂), which is reactive. The oxygen reacts with the cancerous cells and causes cytotoxic oxidation reactions, thereby destroying the cancerous cells. Only biological material that is simultaneously exposed to the photosensitizing agent and light, in the presence of oxygen, are subjected to these reactions.

Environmentally inactive nanoparticles such as those presented for biomedical uses can be used in a variety of other applications that are external to the human body but in which contact with humans or other animals is likely to occur. Examples of such applications include the use of nanoparticles in compositions to cover surfaces (e.g., paints, lacquers, varnishes, sealants, and the like). In one such example, the composition may be used to create messages that are visible under certain conditions. The composition may be used as a coating to protect or decorate a surface. The composition may be transparent or contain pigmentation. The nanoparticles are suspended in the solution of the composition. In one embodiment, the composition may dry to form a hard coating.

Visible fluorescent compositions, such as paints, containing nanoparticles have the same color under visible (white) light as they do under long wavelength (black) or short wavelength (UV) light and are stable under outdoor conditions, thus permitting the compositions to be used in traffic signs for reflection at night, for example. Transparent compositions containing nanoparticles that fluoresce under black or UV radiation can be used to create hidden images. The stability of the nanoparticles permit the compositions to be exposed to sunlight as well as other harsh environmental factors without bleaching. The compositions can be water soluble, if desired.

Nanoparticles may also be used as photocatalysts for providing “self-sterilizing” surfaces, surface coatings, and the like. By way of example, one or more of the nanoparticles may be added to the composition to serve as a catalyst for degrading environmental contaminants including but not limited to dirt, bacteria, fungi, viruses, spores, toxins, chemical warfare agents, biological warfare agents, and the like that may come in contact with the surface.

Accordingly, a composition for covering a surface includes a solution, one or a plurality of pigments, and one or more of the nanoparticles. In some embodiments, at least a portion of the nanoparticles absorb visible light, which may be useful in applications for preventing or minimizing the growth and/or accumulation of environmental contaminants on the exterior surface of a building and/or on interior surfaces for which sterility is desirable (e.g., the walls of a surgical operating room or the like). In some embodiments, the nanoparticles absorb light in the yellow to orange range, which is a particularly acceptable color range for use in paints, coatings, and the like.

Nanoparticles may be used to detect an analyte in a fluid or otherwise interact with a biological organism (cells, neurons, etc . . . ) by coupling to a receptor which interacts with the analyte/organism to the nanoparticles.

Nanocatalysts containing the nanoparticles demonstrate high reactivity under visible light, allowing more efficient usage of light. In the process of semiconductor based photocatalysis, upon absorption of light energy equal to or larger than the band gap energy, a valence band electron of the semiconductor may be excited to the conduction band, leaving a positive hole in the valence band. The positive hole is a strong oxidant, which can either oxidize a compound directly or react with electron donors like water or hydroxide ions to form hydroxyl radicals (.OH), which are also potent oxidizers.

Nanoparticles may be used in photocatalytic or chemical applications such as fuel cell applications or for thermoelectric applications since the nanoparticles are relatively stable when charged with charged carriers. A method for catalyzing a chemical reaction with light includes (a) providing one or more of the nanoparticles; (b) activating at least a portion of the nanoparticles with light to form activated nanoparticles; and (c) catalyzing a chemical reaction with the activated nanoparticles. In some embodiments, the chemical reaction is a transformation selected from the group consisting of conversion of hydrogen gas into protons, conversion of protons and oxygen into water, and a combination thereof. In some embodiments, the chemical reaction occurs in a fuel cell. Fuel cells using nanoparticles include an anode, a cathode containing the nanoparticles in a binder, a separator between anode and cathode, and an electrolyte. Electrons flow from the cathode to the anode. The anode is formed from any material that is suitable for use with the desired electrolyte. The separator is an electrically insulating material permits types of ions to flow therethrough. The nanoparticles may be used in multi-valued logic applications if they exhibit quantized charging or sensor applications if they exhibit reversible charge transference, for example repetitive electrode potential cycling or pulsing between oxidation and reduction.

In summary, single and multiple source precursor methods of producing nanoparticles are described. The nanoparticles contain at least a group I metal and a group VI non-metal element, for example selenium or tellurium, and may contain additional elements, such as those from a group III, IV, or V non-metal. The nanoparticles may be used in many disparate applications including, for example, photovoltaics, lighting applications, displays, imaging or therapeutic applications such as photodynamic therapy (PDT), paints or other compositions to cover a surface, memory devices, sensors, or fuel cells such as batteries.

It is therefore intended that the foregoing detailed description be regarded as illustrative rather than limiting, and that it be understood that it is the following claims, including all equivalents, that are intended to define the spirit and scope of this invention. Nor is anything in the foregoing description intended to disavow scope of the invention as claimed or any equivalents thereof.

### DEFINITIONS

The term “nanoparticle” refers to a particle that exhibits one or more properties not normally associated with a corresponding bulk material (e.g., quantum optical effects, etc.). As used herein, the term usually refers to materials having nanometer-sized dimensions that do not exceed about 1000 nm (although in many embodiments these dimensions are even smaller). In some embodiments, the nanoparticles are in a crystalline state. In some embodiments, the nanoparticles may be used in photocatalytic applications.

The terms “main group,” “transition metal,” “group IVA metals,” “group VA metals,” “group VIA metals,” “group VIIA metals,” “group VIIIA metals,” “group IB metals,” and “group IIB metals” refer to elements as they are grouped in the Periodic Table of the Elements. The subgroup designations A and B refer to the designations recommended by the International Union of Pure and Applied Chemistry (IUPAC).

The phrase “alkyl group” refers to any straight, branched, cyclic, acyclic, saturated or unsaturated carbon chain. Representative alkyl groups include but are not limited to alkanes, alkenes, alkynes, cycloalkanes, cycloalkenes, cycloalkynes, aryls, and the like, and combinations thereof.

The term “transition metal” refers to an element selected from the group consisting of group IVA metals, group VA metals, group VIA metals, group VIIA metals, group VIIIA metals, group IB metals, group IIB metals, and combinations thereof.

Group I metals include copper, silver, gold, zinc, cadmium and mercury. Group VI non-metals include oxygen, sulfur, selenium, and tellurium. Group III non-metals include boron, aluminum, gallium, and indium. Group IV elements include carbon, silicon, germanium, tin, and lead. Group V elements include nitrogen, phosphorous, arsenic, antimony and bismuth.

1. A plurality of nanoparticles comprising:
an element selected from a group I metal;
an element selected from a group VI non-metal; and
an element selected from the group consisting of a group III non-metal a group IV non-metal and a group V non-metal.

2. The nanoparticles of claim 1 forming a portion of an emission layer of a light emitting diode, the light emitting diode further comprising an anode, a cathode and the emission layer therebetween.

3. The nanoparticles of claim 1 forming an excitation source of a coherent light source, the coherent light source further comprising an optical cavity that directs light produced by the nanoparticles and a mirror that directs the light.

4. The nanoparticles of claim 1 forming a portion of a light source of a display, the display further comprising a pair of substrates with a layer whose optical properties change with an applied electric field with the light source supplying light thereto.

5. The nanoparticles of claim 1 forming a portion of a floating gate of a memory device, the memory device further comprising a source, a drain, a channel between the source and the drain, the floating gate above the channel, a control gate above the floating gate, and an oxide layer between the control gate and the floating gate.

6. The nanoparticles of claim 1 forming a portion of a binder in a cathode of a fuel cell, the fuel cell further comprising an electrically insulating separator between an anode and the cathode, and an electrolyte through which electrons flow between the anode and the cathode.

7. The nanoparticles of claim 1 forming a portion of a solvent within a composition to cover a surface.

8. The nanoparticles of claim 7, wherein the composition to cover a surface further comprises a pigment that reflects light in a visible region of the electromagnetic spectrum.

9. A plurality of nanoparticles comprising:
an element selected from a group I metal; and
an element selected from the group consisting of selenium and tellurium.

10. The nanoparticles of claim 9 forming a portion of an emission layer of a light emitting diode, the light emitting diode further comprising an anode, a cathode and the emission layer therebetween.

11. The nanoparticles of claim 9 forming an excitation source of a coherent light source, the coherent light source further comprising an optical cavity that directs light produced by the nanoparticles and a mirror that directs the light.

12. The nanoparticles of claim 9 forming a portion of a light source of a display, the display further comprising a pair of substrates with a layer whose optical properties change with an applied electric field with the light source supplying light thereto.

13. The nanoparticles of claim 9 forming a portion of a floating gate of a memory device, the memory device further comprising a source, a drain, a channel between the source and the drain, the floating gate above the channel, a control gate above the floating gate, and an oxide layer between the control gate and the floating gate.

14. The nanoparticles of claim 9 forming a portion of a binder in a cathode of a fuel cell, the fuel cell further comprising an electrically insulating separator between an anode and the cathode, and an electrolyte through which electrons flow between the anode and the cathode.

15. The nanoparticles of claim 9 forming a portion of a solvent within a composition to cover a surface.

16. The nanoparticles of claim 15, wherein the composition to cover a surface further comprises a pigment that reflects light in a visible region of the electromagnetic spectrum.

17. A solution containing a plurality of nanoparticles comprising:
an element selected from a group I metal; and
an element selected from a group VI non-metal, wherein an average separation between adjacent nanoparticles in the solution is at least about 10 nm.

18. The solution of claim 17, wherein the nanoparticles further comprise an element selected from the group consisting of a group III non-metal element, a group IV non-metal element, and a group V non-metal element.

19. The solution of claim 18, wherein the group VI element is selected from the group consisting of selenium and tellurium.

20. The solution of claim 18, wherein the average separation between adjacent nanoparticles in the solution is at least about 100 nm.

21-54. (canceled)
